(19) United States
(12) Patent Application Publication
(10) Pub. No.: US 2005/0042663 A1
(43) Pub. Date: Feb. 24, 2005

(54) Rule-based modeling of biochemical networks

(76) Inventors: Michael L. Blinov, Los Alamos, NM (US); James R. Faeder, Santa Fe, NM (US); William S. Hlavacek, Santa Fe, NM (US)

Correspondence Address: UNIVERSITY OF CALIFORNIA;LOS ALAMOS NATIONAL LABORATORY, P.O. BOX 1663, MS A187, LOS ALAMOS, NM, 87545, US

(21) Appl. No.: 10/922,703

(22) Filed: Aug. 19, 2004

Related U.S. Application Data
(60) Provisional application No. 60/496,134, filed on Aug. 19, 2003.

(51) Int. Cl. C12Q 1/68; G06G 7/48; G06G 7/58

(52) U.S. Cl. 435/6; 703/11

(57) ABSTRACT

A method for the automatic generation of mathematical/computational models that account comprehensively and precisely for the full spectrum of chemical species implied by user-specified activities, potential modifications and interactions of the molecular components of biomolecules is described. A computer-implemented system that includes software was used to generate models. The software has a user interface that allows a user to generate new models and modify existing models.

### RELATED APPLICATIONS

This application claims the benefit of U.S. Provisional Patent Application 60/496,134, filed Aug. 19, 2003, incorporated by reference herein.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates to a computational method for modeling biochemical networks in cellular systems.

### Computer Program Compact Disk Appendix

One embodiment of the present invention is contained in the computer program compact disk, two copies of which are attached. The contents of the compact disk are incorporated by reference herein for all purposes.

| | |
|---|---|
| Machine Format | IBM PC |
| Operating System | UNIX |

Files

| File Name | Date Created | File Size |
|---|---|---|
| bionetgen.pl | Aug. 18, 2004 | 53 Kbytes |
| fceri_dimer_text.groups | Aug. 18, 2004 | 28 Kbytes |
| fceri_dimer.net | Aug. 18, 2004 | 93 Kbytes |
| fceri_dimer.info | Aug. 18, 2004 | 4 Kbytes |
| fceri_dimer.in | Aug. 18, 2004 | 5 Kbytes |
| fceri_dimer.groups | Aug. 18, 2004 | 7 Kbytes |
| fceri_dimer_text.net | Aug. 18, 2004 | 200 Kbytes |

The contents of the compact disks are subject to copyright protection. The copyright owner has no objection to the reproduction of the contents of the compact disk from the records of the U.S. Patent and Trademark Office, but otherwise reserves all copyrights whatsoever.

### BACKGROUND OF THE INVENTION

Cell signaling, the process through which cells sense and respond to their environment, involves a vast and continually expanding array of receptors, kinases, adaptor molecules, and phosphorylation sites. Early signaling events triggered by receptors in eukaryotic cells usually involve the formation of large, heterogeneous molecular complexes (Hlavacek, W. S., Faeder, J. R., Blinov, M. L., Perelson, A. S. & Goldstein, B. (2003) Biotechnol. Bioeng. 84, 783-794; and Goldstein, B., Faeder, J. R. & Hlavacek, W. S. (2004) Nat. Rev. Immunol. 4, 445-456, both incorporated by reference herein). A typical signaling protein contains multiple sites that may be reversibly modified (e.g., phosphorylated), bind other proteins or lipids, or regulate the protein's enzymatic activity, giving rise to many different protein states and a combinatorially large number of potential signaling complexes. For example, a protein that contains 10 amino acid residues subject to the activities of kinases and phosphatases theoretically has 2¹⁰=1024 states of phosphorylation. If the protein forms homodimers, the number of chemically distinct phosphorylation states is 524,800, a number that might exceed the total number of proteins in the cell. This combinatorial complexity has been largely ignored by both experimentalists and modelers and is a major barrier to predictive understanding of signal transduction.

Experimental resolution of protein states and complexes is usually limited to a small number of sites and interactions, but rapidly advancing proteomic technologies are likely to provide a wealth of more detailed information about signaling complexes in the near future (see, e.g., Aebersold, R. & Mann, M. (2003) Nature 422, 198-207). A number of studies already confirm that a diverse range of molecular complexes arise during signal transduction (see, e.g., Blagoev, B., Kratchmarova, I., Ong, S., Nielsen, M., Foster, L. & Mann, M. (2003) Nat. Biotechnol. 21, 315-318). Because the full spectrum of protein states and complexes is difficult to enumerate, let alone understand, it is likely that mathematical/computational modeling will play an important role in interpreting such data and assessing the functional significance of specific interactions and complexes.

There is increasing recognition that mathematical modeling can be a valuable interpretive and predictive tool for understanding cell signaling cascades, and researchers who study cell signaling systems are beginning to explore the use of mathematical/computational models of signaling systems to aid experimental investigations (see: Kholodenko B., Demin, O., Moehren, G. & Hoek, J. J. Biol. Chem. 274, 30169-30181; Schoeberl, B., Eichler-Jonsson, C., Gilles, E. D. & Muller, G. (2002) Nat. Biotechnol. 20, 370-375; Resat, H., Ewald, J., Dixon, D. & Wiley, H. (2003) Biophys. J. 85, 730-743; and Hatakeyama, Mariko; Kimura, Shuhei; Naka, Takashi; Kawasaki, Takuji; Yumoto, Noriko; Ichikawa, Mio; Kim, Jae-Hoon; Saito, Kazuki; Saeki, Mihoro; Shirouzu, Mikako (2003) Biochemical Journal. 373, 451-463 (2003), all incorporated by reference herein). In fact, several companies (e.g., ENTELOS™ and GENE NETWORK SCIENCES) provide software tools that model signaling systems to assist pharmaceutical companies in drug development.

Few models of signaling developed so far encompass the breadth of chemical species required to address these questions. Instead, most models, given a particular set of proteins and interactions, are based on assumptions (usually implicit) that exclude the vast majority of possible species from consideration. An example is the model of epidermal growth factor receptor signaling that was developed by Kholodenko et al. (vide supra) and extended by several other groups (Schoeberl, B. et al.; Resat, H. et.; and Hatakeyama, M, et. al., vide supra). The original model includes six proteins and tracks 25 species, but lifting implicit assumptions in the model raises the number to hundreds or thousands of species, depending on mechanistic assumptions, even without the introduction of new rate constants or other parameters. While such models have provided valuable insights into signaling mechanisms, they are generally not suitable for addressing the questions of whether and how signaling networks favor specific complexes (of biomolecules, for example), which requires models that consider the full spectrum of possible chemical species.

Accordingly, it is an object of the present invention to provide a method for generating reaction networks of biomolecular interactions.

It is yet another object of the present invention to provide a method for generating a reaction network that considers a significantly larger list of possible chemical species and interactions among them than previous method.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

In accordance with the purposes of the present invention, as embodied and broadly described herein, the present invention includes a method for generating a biochemical reaction network. The method includes specifying an initial set of chemical species comprising biomolecules and their components in specified states; specifying reaction rules for classes of chemical reactions among the chemical species, each rule specifying the properties of the chemical species that react and the rate law and the properties of any chemical species that are formed from a reaction; applying each reaction rule to those species of the initial set of chemical species that match the specification of the rule, thereby generating a first list of reactions and a first list of chemical species that are formed during these reactions; and applying iteratively each reaction rule to all sets of chemical species that are present after the previous step that match the specification of the rule until specified conditions are met, thereby generating a biochemical reaction network that includes a list of chemical species and a list of reactions among those species.

The invention also includes a reaction network produced according to the above method.

The invention also includes a system for modeling biomolecular interactions. The system includes a reader for reading an input file; a software program for specifying an initial set of chemical species comprising biomolecules and their components in specified states; specifying reaction rules for classes of chemical reactions among the chemical species, each rule specifying the properties of the chemical species that react and the rate law and the properties of any chemical species that are formed from a reaction; applying each reaction rule to those species of the initial set of chemical species that match the specification of the rule, thereby generating a first list of reactions and a first list of chemical species that are formed during these reactions; applying iteratively each reaction rule to all sets of chemical species that are present after the previous step that match the specification of the rule, thereby generating a biochemical reaction network that includes a list of chemical species and a list of reactions among those species; and a user interface allowing a user to provide graphical or text input to the software program.

The invention also includes a method for generating a protein interaction network in a cellular system. The method involves specifying an initial set of chemical species comprising proteins and components of proteins in specified states; specifying reaction rules for classes of reactions that correspond to interactions of proteins, with each rule identifying a rate law for reactions of a particular class, a set of properties, comprising particular states of components of proteins, that distinguish chemical species as reactants in reactions of this class, and a transformation, comprising at least one change in the state of a component, that converts reactants to products in reactions of this class; using each reaction rule to find sets of chemical species among a list of chemical species, including the initial set of chemical species, that are reactants in a reaction based on the properties of reactants identified in the rule; and thereafter using the reaction rule to define a reaction, comprising at least one reactant, at least one product, and a rate law, for each set of chemical species found to be reactants, wherein the products of the reaction are determined by applying the transformation identified in the rule to the reactants; maintaining a list of the reactions defined by applying reaction rules and a list of the chemical species involved in these reactions; and applying the reaction rules to the list of chemical species to find all reactions and chemical species involved in these reactions implied by the rules given the initial set of chemical species, thereby generating a biochemical reaction network that includes chemical species, comprising proteins, and reactions among these species, comprising interactions of proteins.

The invention also includes a method of representing a cellular system of interacting biomolecules. The method includes identifying biomolecules and components of these biomolecules in a system; identifying states of biomolecular components; identifying interactions between the biomolecular components; identifying classes of reactions that can result from the interactions of biomolecular components, including the contexts in which reactions are possible; representing each unique chemical species in the system as a string comprising labels that specify the particular state of each biomolecular component contained in the chemical species; representing groups of chemical species as strings comprising labels, wherein the labels may indicate a range of states of biomolecular components; and specifying a reaction rule for each class of reaction to be considered, with the rule comprising a rate law and strings of two types that together indicate a chemical transformation, the first type identifying reactant chemical species and the second type identifying product chemical species in reactions of the class corresponding to the rule.

The invention also includes a method of on-the-fly generation and simulation of a biochemical reaction network. The method includes specifying an initial set of chemical species with non-zero concentration comprising biomolecules and their components in specified states; specifying the concentrations of the chemical species in the initial set; maintaining a list of chemical species and their corresponding concentrations, initially consisting of the initial set of chemical species and their specified concentrations; specifying reaction rules for classes of reactions, with each rule identifying a rate law for reactions of a particular class, a set of properties, comprising particular states of biomolecular components, that distinguish chemical species as reactants in reactions of this class, and a transformation, comprising at least one change in the state of a component, that converts reactants to products in reactions of this class; using each reaction rule to find sets of chemical species among a list of chemical species, including the initial set of chemical species, that are reactants in a reaction based on the properties of reactants identified in the rule; and thereafter, using the reaction rule to define a reaction for each set of chemical species found to be reactants, wherein the products of the reaction are determined by applying the transformation identified in the rule to the reactants; generating an initial set of reactions by applying the reaction rules to the initial set of chemical species, and thereafter, adding new chemical species identified as products to the list of chemical species and corresponding concentrations with concentrations of new species being assigned zero value; maintaining a list of reactions and their corresponding rates, initially consisting of the reactions identified in step (f) and their rates determined by the associated rate law; performing a stochastic simulation of chemical reaction kinetics and updating the list of concentrations at each step in the simulation until the concentration of at least one chemical species with zero value changes to a positive value; and thereafter, applying the reaction rules to generate all reactions in which at least one of the newly populated chemical species is a reactant and updating the list of reactions and rates and the list of chemical species and concentrations, with new chemical species being assigned zero value; thereafter, repeating the previous step.

### BRIEF DESCRIPTION OF THE DRAWINGS

The accompanying drawings, which are incorporated in and form a part of the specification, illustrate the embodiment(s) of the present invention and, together with the description, serve to explain the principles of the invention. In the drawings:

FIG. 1a-c shows a representation of an embodiment model for the early events in FcεRI signaling induced by a bivalent ligand, which is a covalently cross-linked IgE dimer. FIG. 1a shows the tetrameric FcεRI, which is modeled as three subunits, α, β, and γ. FIG. 1b shows the states of the β and γ chains included in the model; the α subunit can be bound to ligand or unbound (2 states of α); the β subunit can be unphosphorylated or phosphorylated, with or without associated Lyn (4 states of β); and the γsubunit can be unphosphorylated or phosphorylated and the phosphorylated form can be bound to Syk in any of four states of phosphorylation (6 states of γ). FIG. 1c shows three of 300 possible states for cross-linked FcεRI dimers.

FIG. 2 shows the 15 reaction classes and 21 rate constants in the model of FIG. 1. The number of reactions of each class (including forward and reverse reactions separately) is listed in parenthesis.

FIG. 3a-e illustrate some of the declarations used in an input file called “fceri_net.in” that was used with software to generate the model of FIG. 1. Boxes shown include actual text used in the file. FIG. 3a shows declarations of six individual chemical species. FIG. 3b shows multi-state species declaration of 48 individual chemical species that contain one receptor (R). Each of these species is characterized by three domains, which have two, four, and six possible states. FIG. 3c shows a declaration of complexes that contain two receptors (left) and an example of one of the 300 individual chemical species in this class (right). FIG. 3d shows the reaction rule for one of the 15 reaction classes, ligand-receptor binding; this rule implies 24 distinct forward reactions and the same number of reverse reactions. All forward (reverse) reactions are assigned the rate constant k₊₁ (k₋₁). FIG. 3e shows a declaration of an output function, a weighted sum of 98 chemical species concentrations, used to calculate the total concentration of autophosphorylated Syk.

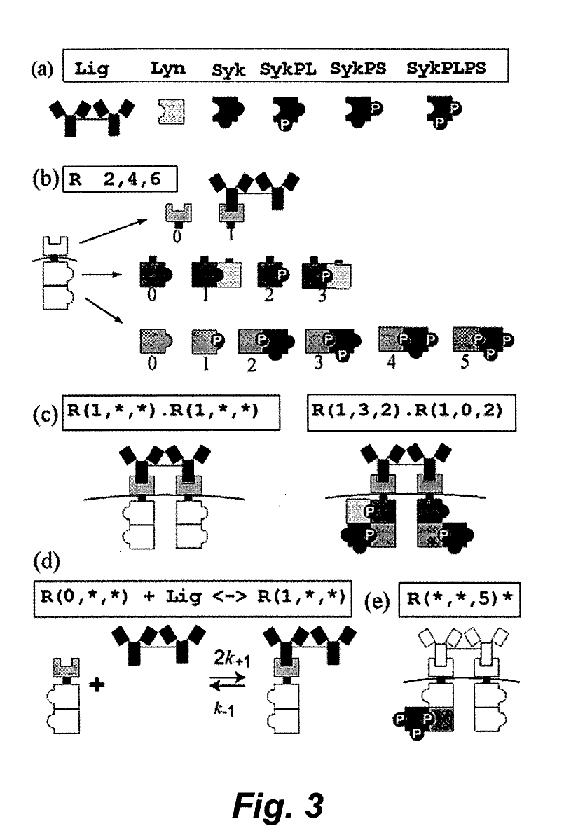

FIG. 4 shows a graph of concentration versus time that includes output data obtained from the method of invention for input file “fceri_net.in”. The data are related to simulated and observed kinetics of phosphorylation and other early signaling events of FcεRI. Each curve corresponds to a set of chemical species having specified features, e.g. all FcεRI receptors having β chain phosphorylated.

### DETAILED DESCRIPTION

Briefly, the invention relates to a method that creates a biochemical reaction network for interacting biomolecules. The network generated using this invention accounts for biomolecules, bimolecular complexes, and other chemical species, that are implied by specified interactions among the biomolecules.

An important aspect of the method relates to creating rules (also referred to herein as reaction rules) for classes of chemical reactions among the chemical species. Each reaction rule specifies the properties required of reacting chemical species, the rate law governing the reactions, and the properties of any chemical species that are formed from the reactions. Each reaction rule is applied to those species that match the specification of the rule. Applying these rules generates a list of chemical reactions and a list of chemical species that participate in the reactions.

The method considers the components of biomolecules, such as binding domains, sites of enzyme-controlled modification, and domains of catalytic activity, and uses reaction rules to generate a biochemical reaction network. The reaction network includes all the possible species and reactions among them that are implied by the reaction rules. Each rule defines a class of reaction that can occur as a result of a set of biomolecular interactions. Application of a rule to each set of chemical species that have the necessary properties of reactants as defined by the rule generates a chemical reaction, which, along with the list of reacting species, includes the list of products formed by the reaction and the kinetic law governing the rate of the reaction.

Software may be used to implement the method and generate biochemical reaction networks of the invention for a wide array of cellular systems. The term “BioNetGen” is used herein to refer to software capable of implementing the invention using a computer. BioNetGen automatically generates all the chemical species and reactions among them that arise from applying a user-defined set of reaction rules to an user-defined initial set of chemical species.

The BioNetGen software is attached to this application in a CD in a file named bionetgen.pl. It provides rule-based modeling of interacting biomolecules found in cellular systems. BioNetGen allows the user to create a biochemical reaction network that characterizes the dynamics of a biomolecular system, accounting for all specified activities and interactions of the components of biomolecules. A model generated with BioNetGen accounts comprehensively and precisely, for example, for specified enzymatic activities and potential post-translational modifications of proteins.

To create a biochemical reaction network with BioNetGen, the user identifies biomolecules, components of the biomolecules, and the possible states of each component. The user also specifies an initial set of chemical species with their concentrations and a set of reaction rules with their associated kinetic parameters.

In addition, the user may specify output functions that sum the concentrations of a set of chemical species with selected properties. This is useful because experimental measurements of biological systems usually do not detect a single chemical species, but rather encompass a broad range of chemical species with a particular biochemical property, such as the total concentration of a protein bound by a phosphotyrosine-specific antibody.

The user-specified information is contained in a single plain text input file. What follows is a more detailed description of the parts of an input file used with the current version of BioNetGen.

The input file may include user-defined parameters that can be used to specify the initial concentrations of chemical species. Each parameter is defined on a separate line in the format
parameter_name value.

The input file must include user-defined rate constants that set values of rate constants used in the definition of reactions. Each parameter is defined on a separate line with the format
rate_constant_name value.

The input file may include a list of single state species with initial concentrations. These are species described as having a single state. Each species is defined on a separate line with the format
species_name initial_concentration.

The input file may include a list of multi-state species. These are species comprised of one or more components, each of which may be in one or more states. Components are not named in the current version of BioNetGen, but are declared by a number that gives the number of states of the component. Each species is defined on a separate line with the format
species_type number_states_component_{—}1 . . . number_states_component_N.

It is important to note that since the state of a component can (and often does) represent the binding of a separate biomolecule, multi-state species may represent complexes of biomolecules. Objects called “complexes”in BioNetGen are defined as two or more multi-state species grouped together to form a single species. These complexes have been used to represent aggregates of biomolecular that are held together through an indirect interaction, such as simultaneous binding to a multi-valent ligand.

The input file may include a list of complexes of multi-state species. Each complex consists of two or more multi-state chemical species bound together. Complexes are assembled through reactions that are defined in reaction rules, but in the current version of BioNetGen, all complexes that may be formed must be explicitly defined in this section because reaction rules that generate previously undefined species are not allowed. Each multi-state species in a complex is specified by a match string of the format name(state_{—}1, . . . , state_N), where name is the name of a multi-state species and each state may be either a number within the range of possible states of the component corresponding to that position or an asterisk, which allows the state of that component to take on any value. A period separates each multi-state component match string in the complex specification. The complex declaration specifies that a new complex should be added to the list of molecular species for each unique set of matching multi-state species. The format of each complex declaration line is
match_string_{—}1 . match_string_{—}2
   where match_string has the format given above.

The input file may include a list of initial concentrations of multi-state species and complexes. Concentrations of multi-state species and complexes if not specified here, are taken by default to be zero. The initial concentration of any multi-state species with its components in specified states or any complex of such multi-state species can be set to a non-zero value through a declaration on this list in the format
species_name value
   Since each component must be in a particular state, using an asterisk is not permitted in the declaration of species_name.

The input file must include a list of reaction rules. Reaction rules specify the transformation of a set of reactant species into a set of product species and rate constants associated with the transformation. Match strings with wildcards (denoted by an asterisk) in the component state positions in the string are used to apply each rule to reactant species with a range of component states. In addition, a wildcard at the end of a match string selects all species containing the specified elements. A period followed by a wildcard at the end of a match string ending selects all complexes containing the specified elements and at least one additional multi-state species. When a match string selecting multiple multi-state species of the same type or ending with a wildcard is applied to a chemical species, the individual match strings for each multi-state species are applied to the multi-state species in a complex in every possible order. Each order that generates a complete match produces a separate reactant configuration to be used in reaction generation, which proceeds by looping over possible reactant configurations. Thus, a rule can generate multiple reactions from the same set of reactant species. More detail on reaction generation is provided in the description of reaction rule processing below.

Transformations of multi-state reactant species are declared by changing the value one or more component states in the specification of the corresponding product. Correspondence between multi-state reactant and product species is established by name and order of appearance. Going from left to right, each multi-state species on the reactant side is assigned a correspondence with the first multi-state species of the same name on product side that has not already been assigned a correspondence. Generally speaking, if a wildcard is used for a component state in a reactant species, a wildcard will also be used to specify the state of the same component in the corresponding product species. Most of the time, a transformation of the state of a component is specified by assigning it a different state in a reactant and its corresponding product. It is permitted to use a wildcard for the state of a reactant component but assign that component a fixed value in the corresponding product, but a reactant component that is assigned a fixed value may not take on a wildcard value in the corresponding product, since such an operation is ill-defined. When a species appears only on the reactant side or only on the product side, the reaction represents a source or a sink term respectively for the species. Reactions may also create, break apart, or change the composition of complexes. Rules to generate reactions only in the forward direction use a forward arrow to separate reactants from products and specify a single rate constant. Rules to generate reactions in both the forward and reverse directions use a bi-directional arrow and specify two rate constants, one for each direction. Specifying a bi-directional reaction rule is equivalent to declaring two unidirectional rules. The rate law for each reaction is taken to be the product of the reactant concentrations and the applicable rate constant. The format of a forward reaction rule is
reactant_{—}1+reactant_{—}2+ . . . −>product_{—}1+ . . . forward_rate_constant
and the format of a reversible reaction rule is
reactant_{—}1+reactant_{—}2+ . . . <−>product_{—}1+ . . . forward_rate_constant, reverse_rate_constant.

The input file may include a list of species groups. Species groups are user-specified lists of species that are used in calculating the aforementioned sums of the species concentrations. The list of species for each group is specified by one or more match strings having the same format as the match strings used in the reaction rules. Two types of sum are allowed. “Species” sums add up the concentration of each species that matches a match string in the specification. “Molecules” sums add up the concentration of each species that matches a match string in the specification weighted by the number of matches found. As mentioned above, match strings ending with a wildcard or containing several multi-state species of the same type can generate multiple matches for the same complex. The format of a species group is
Species group_name match string_{—}1 . . . match_string_N
Molecules group_name match string_{—}1 . . . match_string_N

BioNetGen, which is written in the PERL programming language, translates the high level specification of a model provided in the input file just described, into a biochemical reaction network comprising a list of all possible chemical species and a list of all possible reactions among these species. The BioNetGen program carries out this procedure in the following steps:

Processing user-defined parameters. Names are required to conform to naming conventions and parameters values are required to be non-negative numbers. Names and associated values are checked in a similar manner wherever they appear below. Each name-value pair is stored in a global list.

Processing user-defined rate constants. Each name-value pair is stored in a global list.

Processing single component species. A global list containing species data is initialized, and the name and initial concentration of each single component species is added to the list.

Processing multi-state species. Each multi-state species declaration generates a list of species of the specified type by looping over all possible states of its components. The value of the index corresponding to the state of component i runs from zero to n_states_i−1. The default (first) species generated by multi-state species A with 3 components is A(0,0,0). Looping over components continues from left to right so that the left most component index changes fastest and the right most component index changes slowest. For example, if the first component of A has two possible states, then the first 5 species generated by the declaration “A 2 2 2” are A(0,0,0), A(1,0,0), A(0,1,0), A(1,1,0), and A(0,0, 1). The list of species generated by each multi-state species-declaration is added to the global species list with the corresponding initial concentrations set to zero.

Processing complexes of multi-state species. Each complex declaration is processed as follows. A separate list of matching multi-state species is generated for each match string in the declaration. All match strings must match at least one multi-state species, or an error is returned. A list of new complexes is initialized. Complexes are generated by looping over the multi-state species lists from left to right. For each set of multi-state species in the loop, species of the same type are rearranged into descending sort order without changing the order of other species. Sort order is determined by comparing component state index values of two species of the same type from right to left, with precedence given to a species if it has a higher value at a given position. For example we have
A(1,1)>A(0,1)>A(1,0)>A(0,0).
A string specifying the new complex is generated joining the names of the multi-state species together with periods. If the complex has not been previously added to the list of new complexes, it is added to the list. The loop continues until all combinations have been tried. This procedure avoids generating distinct species for complexes that differ only in the order of their identical elements. For example, A(1,1).A(1,0) and A(1,0).A(1,1) represent identical complexes, so BioNetGen would only generate the first complex.

Processing initial concentrations of multi-state species and complexes. The initial concentration of each multi-state species or complex referenced by a name that appears on the global species list is given the specified initial concentration.

Processing reaction rules. A global list of reactions is initialized and the following steps are carried out for each reaction rule:
(a) Check syntax. The rule is first checked to ensure it conforms to one of the two proper syntaxes given above.
(b) Set the current reaction direction to be forward.
(c) Set the current rate constant to be the first rate constant in the rule if the current reaction direction is forward, the second rate constant in the rule otherwise.
(d) Establish correspondence between match strings of the reactants and products. Match strings specifying complexes are first broken up into match strings for multi-state species and wildcards that represent any unmatched portion of a complex. Correspondence between reactant and product match strings is determined by looping over reactant match strings from left to right assigning a correspondence to the first product match string (going also left to right) of the same type not already assigned. A null correspondence is established if a match is not found.
(e) Determine equivalences of reactant match strings. If two or more reactant match strings are identical (excluding wildcards), they are checked for equivalence. Two reactant match strings in a reaction are defined to be equivalent if and only if (1) the strings are identical, (2) the match strings of their corresponding product match strings are identical, (3) the full match strings of the reactant species in which they appear are identical, and (4) the full match strings of the product species in which their corresponding product match strings appear are identical. The first two tests establish that the match strings select identical multi-state species and that these species undergo identical transformations during the reaction. The second two tests establish that the match strings on both reactant and product sides of the reaction select multi-state species within complexes of identical composition. The equivalence property is used below to ensure that reactions involving multiple species of the same type are generated with the correct multiplicity.
(f) Initialize a list containing the data for reactions arising from this reaction rule applied in the current reaction direction.
(g) For each reactant in the rule, create a list of reactant species using the full match string to select matching species on the global species list.
(h) For each set of reactant species drawn by looping over the reactant species lists:
   1. Go to the next set if the species matching any pair of equivalent match strings is not in sort order. If two match strings within a rule are equivalent, the order in which the matching species appear in the reaction does not affect the reaction. Therefore, by choosing a definite order, the reaction is only generated once.
   2. Go to the next set if a pair of equivalent match strings matches a pair of species within a complex and in an order that does not match the order of the species within the complex. Because the check in this step is performed first and species within a complex appear in sort order, this condition only applies when the matching species are identical. In this case, the match strings can match the species in either order. If the match strings are not equivalent, each order generates an instance of the same reaction, appropriately leading to a reaction with multiplicity 2. When the match strings are equivalent, this check permits only one ordering, appropriately leading to a reaction with multiplicity 1. These cases are illustrated by examples of symmetric and asymmetric breakup of dimers. In the asymmetric example,
      A(1,*).A(1,*)−>A(1,*)+A(0,*)
      the two reactant match strings (A(1,*) on the left and A(1,*) on the right) are not equivalent, because they correspond to different product match strings. When BioNetGen applies the reactant match string A(1,*).A(1,*) to the complex A(1,0).A(1,0), it generates two matches because it checks all possible rearrangements of identical species types within the complex. Thus, BioNetGen would create the reaction
      A(1,0).A(1,0)−>A(1,0)+A(0,0)
      with multiplicity 2, which is correct because either of the two A molecules can undergo a change in the state of component 1 when the dimer breaks apart. In the symmetric example,
      A(1,*).A(1,*)−>A(1,*)+A(1,*)
      the two reactant match strings are equivalent. Thus, when BioNetGen encounters the reactant complex A(1,0).A(1,0) it correctly generates the reaction
      A(1,0).A(1,0)−>A(1,0)+A(1,0)
      with multiplicity 1 because of the check performed in this step. By checking the order in which the match strings match the species in the complex, this step correctly eliminates one instance of the reaction.
   3. Generate list of product species using correspondence between reactant and product species. For each multi-state reactant species selected by a single match string, the corresponding product species is generated by changing the component states to match the component states of the corresponding product match string. Reactant species selected by a single match string that have no corresponding product do not appear in the products. Product match strings that do not have a corresponding reactant match string must specify a single valid species, or an error is returned. The component states of species matched by a wildcard do not change in going from reactant to product. Once all single match strings in the products are associated with species, product complexes are generated according to the groupings specified by full product match strings.
   4. If a reaction with the same reactant and product species does not appear on the current reaction list, add it to the list, setting the corresponding multiplicity to one; otherwise, increment the multiplicity of the existing reaction by one.
(i) Add the current reaction list, in which the rate constant for each reaction is the multiplicity of the reaction multiplied by the current rate constant, to the global list of reactions.
(j) If the rule is to be applied in the reverse direction and the current direction is forward, change the current reaction direction to reverse, swap the arrays containing the match strings for reactants and products, and go to step (c) above.

Processing species groups. A global list containing species group data is initialized. Each group entry generates a new group of the specified name and type and initializes lists of group species and multiplicities. Each match string in the group definition is used to select species from the global species list; the matching species are then used to update the group species and multiplicities. The group type determines whether or not the multiplicities are used in computing a sum of species concentrations.

Writing the network to a file. The network generated by BioNetGen is comprised of the following elements, which are written to an output file with the extension net:
(a) List of parameters. The format of each list entry is
   parameter_name value
(b) List of rate constants. The format of each list entry is
   rate_constant_name value
(c) List of species. The format of each list entry is
   species_name initial_concentration
(d) List of reactions. The format of each list entry is
   react_spec_{—}1, . . . ,react_spec_N prod_spec_{—}1, . . . ,prod_spec_N multiplicity*rate_const_name
(e) List of species groups. (optionally written to a separate file with the groups extension). The format of each list entry is
   group_name multiplicity_{—}1*species_{—}1, . . . ,multiplicity_N*species_N
      Multiplicities are omitted for groups of type Species.

The output of BioNetGen can be used to predict the properties of the biochemical reaction network. For that, a reaction network generated by BioNetGen can be read by other programs. These include a program called ‘Network’ that translates the reaction network into a set of coupled ordinary differential equations (ODEs) and solves the ODEs (see http://cellsignaling.lanl.gov/354/for the details of ‘Network’). ‘Network’ sends the time-courses of concentrations and output functions in tabular format to files that can be imported into visualization software, such as GRACE (http://plasma-gate.weizmann.ac.il/Grace), for which an interface is provided by BioNetGen software. BioNetGen also exports models in systems biology markup language (SBML) format (see M. Hucka et al., “The Systems Biology Markup Language (SBML): A Medium for Representation and Exchange of Biochemical Network Models,” Bioinformatics, vol. 19, pp. 524-531 2003, incorporated by reference herein). As a result, models generated with BioNetGen are usable by the various software tools that support SBML (see: http://sbml.org). These tools include programs that implement discrete-event Monte Carlo algorithms for simulating stochastic chemical reaction kinetics (see: D. T. Gillespie, “A General Method for Numerically Simulating The Stochastic Time Evolution of Coupled Chemical Reactions,” J. Comp. Phys., vol. 22, pp. 403-434, 1976, incorporated by reference).

The conventions used for the BioNetGen input file provide a concise language for specifying models that account for the modifications and interactions of molecular domains. For example, the input file that specifies the model to be described later includes 30 reaction rules and requires 5 kilobytes of memory. In contrast, the SBML file that specifies this model requires the explicit declaration of 3,680 unidirectional reactions and is more than a megabyte in size (because of both the verbose XML encoding and the number of reactions).

Having thus provided a general description of the invention and of BioNetGen; a detailed example relating to using the invention and BioNetGen to study signaling by FcεRI, which is high affinity receptor for IgE antibody, now follows (see U.S. Provisional Patent Application 60/496,134, filed Aug. 19, 2003; J. R. Faeder et. al., “Investigation of Early Events in FcεRI-Mediated Signaling Using a Detailed Mathematical Model,” Journal of Immunology, vol. 170, pp. 3769-3781, 2003, incorporated by reference herein; B. Goldstein et al., “Modeling the Early Signaling Events Mediated by FcεRI,” Molecular Immunology, vol. 38, pp. 1213, incorporated by reference herein; B. Goldstein et al. 2004; and W. S. Hlavacek et al., vide supra). This receptor is found in rodent cells and is also expressed on human basophils and mast cells.

Some abbreviations are used throughout. Some of them are: ITAM (immunoreceptor tyrosine-based activation motif); PTK (protein tyrosine kinase); SH2 (Src homology 2).

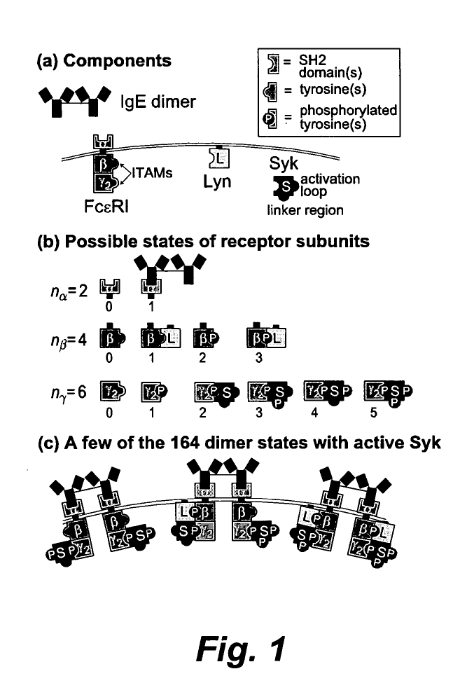

The invention may be better understood using the accompanying figures. As FIG. 1 shows, FcεRI is divided into the following components: an α subunit that binds IgE, a β subunit, and a disulfide-linked dimer of γ subunits. The ≢ and γ subunits contain ITAMs that become phosphorylated on tyrosine residues. The membrane-associated kinase Lyn can associate with the unphosphorylated or phosphorylated β subunit. The cytosolic kinase Syk associates with a doubly phosphorylated γ ITAM. On the representations of the kinases, notches indicate SH2 domains. The bumps on β subunits, γ subunits, and Syk kinase represent groups of tyrosines that are lumped together in the model as single targets for phosphorylation. On Syk, the model distinguishes tyrosines in the linker region, which are phosphorylated by Lyn, from those in the activation loop, which are phosphorylated by Syk.

The model generated using BioNetGen is based on the following sequence of early events in signaling through the FcεRI receptor: ligand-receptor binding leading to aggregation of receptors at the plasma membrane, transphosphorylation of specific tyrosine residues in the ITAMs of aggregated receptors by constititutively associated Lyn kinase. The β subunit and the γ dimer upon phosphorylation become binding sites the two kinases, Lyn and Syk; Syk can be subsequently transphosphorylated by both Lyn and Syk.

FIG. 1b shows the states of the β and γ chains included in the model; the γ subunit can be bound to ligand or unbound (2 states of α); the β subunit can be unphosphorylated or phosphorylated, with or without associated Lyn (4 states of β); and the y subunit can be unphosphorylated or phosphorylated and the phosphorylated form can be bound to Syk in any of four states of phosphorylation (6 states of γ). Since the state of each subunit is independent of the states of the other subunits, the total theoretically possible number of monomer states is the product of numbers of states for each subunit, which in this case is 48. In a receptor dimer, each α subunit must be engaged with the ligand, so the total theoretically possible number of dimers is 300.

FIG. 1c shows three of 300 possible states for cross-linked FcεRI dimers that are generated by BioNetGen. In addition, there are six non-receptor states specified in the input file—free ligand, free Lyn, and Syk in each of its four possible states of phosphorylation—to give a total of 354 distinct chemical species in the reaction network generated by BioNetGen.

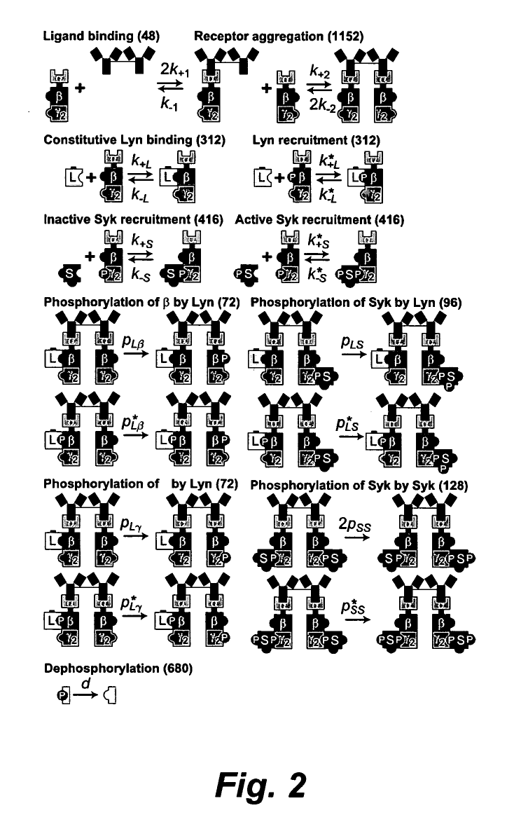

The reaction network is composed of the chemical species that were just described and the chemical reactions that connect them. The reaction network is generated by BioNetGen using a set of reaction rules or classes, each of which describes a chemical interaction based only on the presence of components in specified states of the chemical species involved. For example, as illustrated in FIG. 2, there are only two classes of reaction to describe the formation of a ligand-receptor bond, depending only on whether IgE dimer is already bound to receptor. Since there is experimental evidence that the ligand-receptor interactions in this system are unaffected by modifications of the cytoplasmic portion of the receptor, we assume that rate constants for binding reactions are independent of the states of β and γ subunits. As a result, only two rate constants parameterize the 48 reactions (considering forward and reverse reactions separately) for the binding of free IgE dimer to FcεRI. Similarly, only two rate constants parameterize the far greater number of aggregation reactions (1152) where a free receptor binds to an IgE dimer that is already bound to a receptor. In the current model, BioNetGen uses 15 reaction classes and 21 rate constants (which are described in the file fceri_dimer.in attached to this application on a CD), to generate the 3680 chemical reactions that can occur (which are described in the files fceri_dimer.net, fceri_dimer_text.net, and fceri_dimer.info, attached to this application on a CD). In this way, the limited experimental information available about the kinetics of signaling reactions can be combined with the much larger amount of information available about the structure of the components and the interactions among them.

Each reaction class is illustrated in FIG. 2 by its simplest member. The reactants shown have the minimal set of modifications required to carry out the given reaction. BioNetGen generates the full list of 3680 possible reactions. The rate law for each reaction is generated as the rate constant, which is specified in the input file, multiplied by each of the reactant concentrations (elementary reaction kinetics). The last reaction class in FIG. 2 indicates that phosphorylated units that are not protected through association with an SH2 domain can be dephosphorylated, with a common rate constant, d.

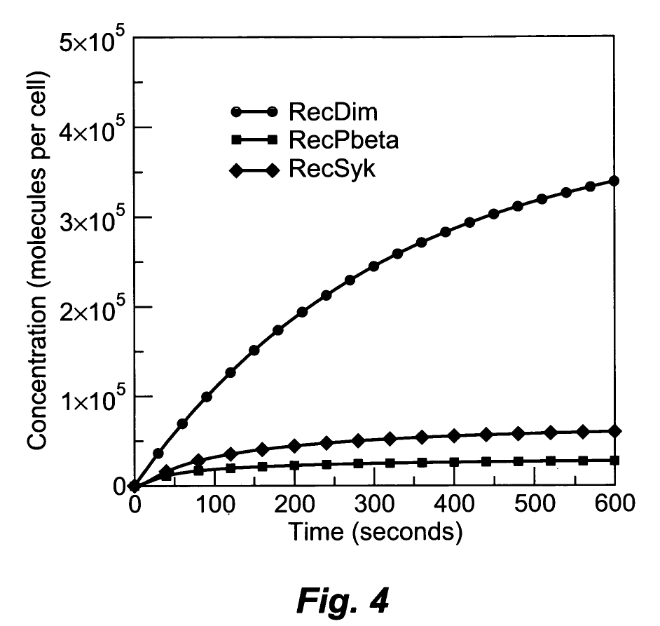

The generated reaction network along with the numeric rate parameters and initial concentrations is passed to a program such as ‘Network’, which is capable of generating and solving a mathematical model that includes of 354 differential equations for the concentration of each chemical species as a function of time. To compare simulation results with experiments, the concentrations of species with a particular characteristic, e.g. phosphorylated γ ITAM, can be lumped (i.e. added) together, as illustrated the files fceri_dimer.groups and fceri_dimer_text.groups that are attached to this application on a CD. FIG. 4 shows a graph of concentration versus time that includes output data obtained from the method of invention for input file “fceri_net.in”. The data are related to simulated and observed kinetics of phosphorylation and other early signaling events of FcεRI. Each curve corresponds to a set of chemical species having specified features, e.g. all FcεRI receptors having β chain phosphorylated.

One advantage of the invention that should be appreciated is related to the new information made available by analyzing the model generated using this invention, which has not been provided by analyzing models obtained using other methods. The model makes predictions as a function of time, ligand concentration, concentrations of signaling components, and potential experimental modifications of the interacting proteins (e.g. removal or impairment of a domain). The last area is particularly important because much of what is known about signaling pathways is derived from the genetic manipulation of the components. The model provides a tool for interpreting such experiments and designing new ways to use genetically altered proteins to test ideas about molecular interactions and signaling mechanisms. The model was also used to identify additional experiments that may be used to improve estimates of key parameters. Through a comparison of model predictions with data, parameter estimates may be improved. Mechanistic assumptions that were put in as a basis for rules specifications were tested by comparing model predictions with previously measured experimental data. The invention was also used to investigate the separate roles of the receptor components (subunits) and the kinases Lyn and Syk.

Several types of results are provided by the analysis of the model generated with BioNetGen: tests of mechanistic assumptions, estimation of unknown parameters, and additional tests to see if model predictions based on the estimated parameters are quantitatively consistent with available data. It was shown that the model is quantitatively consistent with experimental data on the kinetics of phosphorylation of receptor subunits and Syk, dose-response relations, kinetics of dephosphorylation of receptor subunits, and differences between the γ and β subunits in all of these types of experiments. The model was used to predict changes in system behavior in experiments where particular system parameters are altered. The model predicts behavior indicative of “kinetic proofreading” that is consistent with experimental observations.

Another use of the invention relates to testing models generated by other methods and to determine conditions under which they make valid predictions and also to determine the conditions when the predictions may be misleading.

One aspect of the invention relates to identifying new experiments to test biological ideas. Analysis of models generated using this invention allows identification of additional experimental measurements and qualitative manipulations for distinguishing mechanisms and providing accurate quantitative predictions.

Most software tools for modeling signal transduction require a user to make a declaration of some type for each species and reaction in a model, which is a severe limitation for systems marked by combinatorial complexity. By contrast, BioNetGen enables a user to specify a small number of rules to generate a large reaction network.

New species and reactions may be generated using this invention ‘on-the-fly’ (see W. S. Hlavacek et al., vide supra). The term ‘on-the-fly’ is meant to involve simulating the reaction dynamics during the generation of the biochemical reaction network from the reaction rules. A method for simultaneous network generation and Monte Carlo simulation of reaction dynamics for chemical systems of organic molecules described by the Dugundji-Ugi model has been reported (see: Jean-Loup Faulon and Allen G. Sault, 2001, J. Chem. Inf. Comput. Sci., vol. 41, pp. 894-908). The reaction generator used by Faulon and Sault is replaced by the rule-based reaction generator for biomolecular systems of this invention described herein.

In summary, the method of the invention uses rules to generate models of biomolecular systems. These models can be used to guide biological and biomedical research, and pharmaceutical drug development, by generating models that include known drugs, drug candidates, lead compounds and other materials that have or are suspected to have pharmaceutical activity. The models generated using this invention may be relevant for drug discovery, analysis of proteomic data, mechanistic studies of signal transduction, and other important applications.

The foregoing description of the invention has been presented for purposes of illustration and description and is not intended to be exhaustive or to limit the invention to the precise form disclosed, and obviously many modifications and variations are possible in light of the above teaching. For example, the invention has been used to generate other models besides the model for FcεRI signaling described herein; models have also been generated for early membrane-proximal signaling events triggered by epidermal growth factor, erythropoietin, interleukin-1, and for mitogen-activated protein kinase cascades (see http://cellsignaling.lanl.gov/bionetgen).

The embodiment(s) were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

1. A method for generating a biochemical reaction network comprising:
(a) specifying an initial set of chemical species comprising biomolecules and their components in specified states;
(b) specifying reaction rules for classes of chemical reactions among the chemical species, each rule specifying the properties of the chemical species that react and the rate law and the properties of any chemical species that are formed from a reaction;
(c) applying each reaction rule to those species of the initial set of chemical species that match the specification of the rule, thereby generating a first list of reactions and a first list of chemical species that are formed during these reactions; and
(d) applying the reaction rules iteratively to all sets of chemical species that are present after the previous step that match the specification of the rule until specified conditions are met, thereby generating a biochemical reaction network that includes a list of chemical species and a list of reactions among those species.

2. The method of claim 1, wherein the chemical species are selected from the group consisting of proteins, lipids, nucleic acids, drugs, artificially engineered molecules, drug candidates, and lead compounds.

3. The method of claim 1, wherein the components of the biomolecules are selected from the group consisting of proteins, polypeptide chains, amino acids, nucleic acids, sugars, fatty acids, ATP, ADP, AMP, GTP, GDP, amino acid residues subject to post-translational modification, protein or nucleic acid sequence motifs, binding domains of proteins, and catalytic domains of proteins.

4. The method of claim 1, wherein the components of the biomolecules include functional domains comprising enzymatic subunits and sites of post-translational modification.

5. The method of claim 1, wherein the chemical species and reaction classes are selected from the group of biological processes consisting of cell growth, cell proliferation, cell differentiation, cell death, cell-cell communication, protein cleavage, protein degradation, lipid degradation, nucleic acid degradation, and regulation of gene expression.

6. The method of claim 1, wherein the properties of the chemical species are selected from the group consisting of enzymatic activities, conformations, post-translational modifications, and enzyme-catalyzed modifications of biomolecules.

7. The method of claim 1, further comprising:
(e) specifying initial concentrations for the initial set of chemical species; and
(f) expressing the biochemical reaction network as a system of ordinary differential equations that define changes in concentrations of the chemical species over time.

8. The method of claim 7, further comprising:
(g) using the system of ordinary differential equations to predict the behavior of the biochemical reaction network.

9. The method of claim 1, further comprising:
(e) using the list of reactions to predict the properties of the biochemical reaction network

10. The method of claim 1, further comprising:
(e) using the list of species and reactions to track transformations of biomolecules and their components through the reaction network.

11. The method of claim 1, further comprising:
(e) specifying output rules, each output rule identifying a group of species with particular properties.

12. The method of claim 8, further comprising:
(h) specifying output rules, each output rule identifying a group of species with particular properties;
(i) finding chemical species with the properties specified by the output rules;
(j) specifying a mathematical function of the concentrations of the species in the groups; and
(k) evaluating the function.

13. A biochemical reaction network prepared by the method comprising:
(a) specifying an initial set of chemical species comprising biomolecules and their components in specified states;
(b) specifying reaction rules for classes of chemical reactions among the chemical species, each rule specifying the properties of the chemical species that react and the rate law and the properties of any chemical species that are formed from a reaction;
(c) applying each reaction rule to those species of the initial set of chemical species that match the specification of the rule, thereby generating a first list of reactions and a first list of chemical species that are formed during these reactions; and
(d) applying iteratively each reaction rule to all sets of chemical species that are present after the previous step that match the specification of the rule until specified conditions are met, thereby generating a biochemical reaction network that includes a list of chemical species and a list of reactions among those species.

14. A computer system for generating biochemical reaction networks comprising:
a reader for reading an input file;
a software program for specifying an initial set of chemical species comprising biomolecules and their components in specified states; specifying reaction rules for classes of chemical reactions among the chemical species, each rule specifying the properties of the chemical species that react and the rate law and the properties of any chemical species that are formed from a reaction; applying each reaction rule to those species of the initial set of chemical species that match the specification of the rule, thereby generating a first list of reactions and a first list of chemical species that are formed during these reactions; applying iteratively each reaction rule to all sets of chemical species that are present after the previous step that match the specification of the rule until specified conditions are met, thereby generating a biochemical reaction network that includes a list of chemical species and a list of reactions among those species; and
a user interface allowing a user to provide graphical or text input to said software program.

15. A method for generating a protein interaction network in a cellular system, comprising:
(a) specifying an initial set of chemical species comprising proteins and components of proteins in specified states;
(b) specifying reaction rules for classes of reactions that correspond to interactions of proteins, with each rule identifying a rate law for reactions of a particular class, a set of properties, comprising particular states of components of proteins, that distinguish chemical species as reactants in reactions of this class, and a transformation, comprising at least one change in the state of a component, that converts reactants to products in reactions of this class;
(c) using each reaction rule to find sets of chemical species among a list of chemical species, including the initial set of chemical species, that are reactants in a reaction based on the properties of reactants identified in the rule; thereafter,
(d) using the reaction rule to define a reaction, comprising at least one reactant, at least one product, and a rate law, for each set of chemical species found to be reactants, wherein the products of the reaction are determined by applying the transformation identified in the rule to the reactants;
(e) maintaining a list of the reactions defined by applying reaction rules and a list of the chemical species involved in these reactions; and
(f) applying the reaction rules to the list of chemical species to find all reactions and chemical species involved in these reactions implied by the rules given the initial set of chemical species, thereby generating a biochemical reaction network that includes chemical species, comprising proteins, and reactions among these species, comprising interactions of proteins.

16. A method of representing a cellular system of interacting biomolecules, comprising:
(a) identifying biomolecules and components of these biomolecules in a system;
(b) identifying states of biomolecular components;
(c) identifying interactions between the biomolecular components;
(d) identifying classes of reactions that can result from the interactions of biomolecular components, including the contexts in which reactions are possible;
(e) representing each unique chemical species in the system as a string comprising labels that specify the particular state of each biomolecular component contained in the chemical species;
(f) representing groups of chemical species as strings comprising labels, wherein the labels may indicate a range of states of biomolecular components; and
(g) specifying a reaction rule for each class of reaction to be considered, with the rule comprising a rate law and strings of two types that together indicate a chemical transformation, the first type identifying reactant chemical species and the second type identifying product chemical species in reactions of the class corresponding to the rule.

17. The method of claim 16, further comprising:
(h) specifying an initial set of chemical species;
(i) using a reaction rule to identify sets of chemical species that qualify as reactant chemical species;
(j) defining a reaction for each set of reactant chemical species, with the definition comprising the reactant chemical species, the rate law of the reaction rule for this class of reaction, and the product chemical species, which are generated by the transformation defined in the reaction rule;
(k) maintaining a list of all reactions that are found by applying reaction rules;
(l) maintaining a list of chemical species consisting of the initial set of chemical species and product chemical species that are found by applying reaction rules;
(m) applying reaction rules to a list of chemical species to find reactions and products of these reactions; and
(n) repeating the application of reaction rules when product chemical species are found that are not in the list of chemical species available before applying the rules.

18. The method of claim 17, further comprising:
(O) converting the lists of reactions and chemical species into a bipartite graph that represents a biochemical reaction network, in which one type of node corresponds to chemical species and the other type of node corresponds to reactions and directed edges join nodes, indicating the chemical species that participate in each reaction.

19. The method of claim 17, further comprising:
(O) converting the lists of reactions and chemical species into a system of coupled ordinary differential equations (ODEs), in which the independent variable is time, the time-dependent variables are concentrations of the chemical species, the left-hand side of each equation is the time rate of change of the concentration of a particular chemical species, and the right-hand side of each equation is a sum of terms, in which positive terms correspond to the rate laws of reactions that produce the chemical species and negative terms correspond to the rate laws of reactions that consume the chemical species; and
(p) specifying initial values of the concentrations of chemical species.

20. The method of claim 17, further comprising:
(O) specifying output rules, each defining a group of chemical species comprising components of biomolecules in particular states and a function of the properties of these chemical species;
(p) finding sets of chemical species that correspond to groups defined in the output rules; and thereafter,
(q) evaluating the functions defined in output rules.

21. The method of claim 17, further comprising:
(O) writing the list of reactions in the format of the systems biology markup language (SBML); and
(p) using the SBML encoding of the list of reactions and a software package that recognizes SBML to perform Monte Carlo simulation of stochastic chemical kinetics, integration of ordinary differential equations, or evaluation of the vector field of a system of ordinary differential equations.

22. The method of claim 16, wherein strings used to represent chemical species are replaced by graphs, in which nodes of a graph represent biomolecular components, which are indicated by labels of the nodes, and edges of a graph represent bonds between biomolecular components; and wherein strings used to represent groups of chemical species are replaced by graphs, which indicate the common properties of chemical species belonging to a group.

23. The method of claim 17, wherein chemical species and groups of chemical species are represented by graphs.

24. The method of claim 17, wherein the strings are representations of graphs, such that there is a one-to-one correspondence between each string and a graph.

25. A method of on-the-fly generation and simulation of a biochemical reaction network, comprising:
(a) specifying an initial set of chemical species with non-zero concentration comprising biomolecules and their components in specified states;
(b) specifying the concentrations of the chemical species in the initial set;
(c) maintaining a list of chemical species and their corresponding concentrations, initially consisting of the initial set of chemical species and their specified concentrations;
(d) specifying reaction rules for classes of reactions, with each rule identifying a rate law for reactions of a particular class, a set of properties, comprising particular states of biomolecular components, that distinguish chemical species as reactants in reactions of this class, and a transformation, comprising at least one change in the state of a component, that converts reactants to products in reactions of this class;
(e) using each reaction rule to find sets of chemical species among a list of chemical species, including the initial set of chemical species, that are reactants in a reaction based on the properties of reactants identified in the rule; and thereafter, using the reaction rule to define a reaction for each set of chemical species found to be reactants, wherein the products of the reaction are determined by applying the transformation identified in the rule to the reactants;
(f) generating an initial set of reactions by applying the reaction rules to the initial set of chemical species, and thereafter, adding new chemical species identified as products to the list of chemical species and corresponding concentrations with concentrations of new species being assigned zero value;
(g) maintaining a list of reactions and their corresponding rates, initially consisting of the reactions identified in step (f) and their rates determined by the associated rate law;
(h) performing a stochastic simulation of chemical reaction kinetics and updating the list of concentrations at each step in the simulation until the concentration of at least one chemical species with zero value changes to a positive value; and thereafter,
(i) applying the reaction rules to generate all reactions in which at least one of the newly populated chemical species is a reactant and updating the list of reactions and rates and the list of chemical species and concentrations, with new chemical species being assigned zero value; thereafter, repeating step (h).
